(19) United States
(12) Patent Application Publication
(10) Pub. No.: US 2009/0304617 A1
(43) Pub. Date: Dec. 10, 2009

(54) LOW RESIDUE ANTIPERSPIRANT STICK

(76) Inventors: Bernhard Banowski, Dusseldorf (DE); Marcus Claas, Hilden (DE); Armin Wadle, Erkrath (DE)

Correspondence Address: Henkel Corporation, 10 Finderne Avenue, Bridgewater, NJ, 08807, US

(21) Appl. No.: 12/492,278

(22) Filed: Jun. 26, 2009

Related U.S. Application Data
(63) Continuation of application No. PCT/EP2007/063652, filed on Dec. 11, 2007.

(30) Foreign Application Priority Data
Dec. 29, 2006 (DE) 10 2006 062 567.6
Dec. 10, 2007 (DE) 10 2007 059 678.4

(51) Int. Cl. A61K 8/92

(52) U.S. Cl. 424/68; 424/65

(57) ABSTRACT

The present invention relates to low residue, water-free antiperspirant sticks with enhanced properties.

### CROSS-REFERENCE TO RELATED APPLICATIONS

The present application is a continuation of International Application No. PCT/EP2007/063652, filed 11 Dec. 2007, which claims the benefit of German Patent Application Nos. 10 2006 062 567.6, filed 29 Dec. 2006 and 10 2007 059 678.4, filed 10 Dec. 2007.

The present invention relates to an antiperspirant stick of the waterless or low-water dispersion stick type, in which the antiperspirant active ingredients are dispersed in a vehicle comprising a liquid apolar vehicle material, solidifying fat or wax components and surfactants.

Various forms of cosmetic preparations are commercially available for inhibiting perspiration and combating body odor. Of these, stick formulations are especially popular because of their easy and practical handling. The basic composition of such stick preparations must provide a certain hardness and dimensional stability on one hand, but also must be able to rub off on the skin under slight pressure. These consistency properties can be achieved, for example, by aqueous alcoholic or aqueous glycolic preparations solidified with soap to form a gel. However, such sticks are disadvantageous in that acidic aluminum salts (e.g., aluminum chlorohydrate) cannot be formulated in the soap gel sticks because they have a strongly basic pH.

So-called anhydrous dispersion sticks have therefore acquired special significance. These sticks consist of a dispersion of finely divided astringent aluminum or zirconium salts in an apolar liquid vehicle solidified by gelatinizers or waxy fat components. For example, hydrocarbons or silicone oils may be used as the apolar liquid vehicles, preferably those with boiling points below 200° C. or with certain volatility (e.g., cyclic siloxanes (cyclomethicones)) and mixtures thereof with skin softening oil components.

Still, these anhydrous dispersion sticks occasionally have the disadvantage that the onset of the antiperspirant effect of the astringent salts is not rapid enough or intense enough. These state-of-the-art sticks also have the undesirable effect of leaving a more or less definite residue on the skin or causing heavy soiling of clothing that comes in contact with the treated area of skin.

Hydrophilization of the stick composition has already been achieved by adding surfactants, which has also improved the washability of residues from the skin or clothing items. For example, U.S. Pat. No. 4,151,272 discloses antiperspirant sticks containing up to 5 wt % of a fatty alcohol polyglycol ether. EP 491843 B1 discloses antiperspirant stick compositions containing an addition product of 10 mol propylene oxide onto butanediol to reduce the whitening effect. EP 777463 B 1 discloses antiperspirant sticks containing 11,12-dihydroxystearic acid as the solidifying fat or wax component.

However, those stick compositions mentioned above still have various disadvantages. For example, stick compositions containing too little of the solidifying wax components tend to separate oil in the heat. The antiperspirant effect of stick compositions in which the liquid vehicle components do not include any polar components is too weak.

Another problem with the known sticks is their high cyclomethicone content. With the usual cyclomethicones, there is a differentiation mainly of cyclotetrasiloxane, cyclopentasiloxane and cyclohexasiloxane. Cyclotetrasiloxane, having an unusually high melting point of −11° C., can result in storage stability problems in the larger use quantities typical of an anhydrous or low-water dispersion stick. Furthermore, cyclotetrasiloxane is not generally used today for toxicological reasons. Conventional commercial products are also free of cyclotetrasiloxane. Nevertheless, the cyclomethicone class of substances is a problematical raw material today because of even trace amounts of cyclotetrasiloxane content. On the other hand, cyclomethicones have excellent use properties, so it is extremely difficult to replace them.

One object of the present invention is to provide an adequate substitute material for cyclomethicones in anhydrous antiperspirant sticks. Another object of the present invention is to provide a highly effective antiperspirant stick having good rub-on properties. Another object of the present invention is to provide a highly effective antiperspirant stick with low soiling properties. Another object of the present invention is to provide an antiperspirant stick, which is still highly effective and has minimized residue behavior.

It has now surprisingly been found that the objects formulated here can be achieved in an excellent manner by antiperspirant sticks, even when they contain a relatively large amount of oil that is liquid under standard conditions and chosen from linear polydimethylsiloxanes having 2 to 50 siloxane units, optionally in combination with at least one oil that is liquid under standard conditions and chosen from poly-C₂-C₁₆-olefins, which are preferably hydrogenated.

The subject of the invention is therefore anhydrous antiperspirant sticks containing
a) at least one lipid or wax component having a melting point of >30° C.,
b) at least one oil that is liquid under standard conditions and is selected from linear polydimethylsiloxanes with 2 to 50 siloxane units;
c) max. 5 wt % water, based on the total composition,
d) at least one antiperspirant active ingredient.

All statements made with respect to the physical states of the starting materials used (solid, liquid, etc.) in this patent application are based on standard conditions. In the present patent application, “standard conditions” refer to a temperature of 20° C. and a pressure of 1013.25 mbar. Melting point data are also based on a pressure of 1013.25 mbar.

### Antiperspirant Active Ingredients

Antiperspirant active ingredients that may be used in antiperspirant sticks according to the present invention include all conventional aluminum salts, zirconium salts and aluminum zirconium salts known to be suitable as antiperspirant active ingredients. These salts include aluminum halides and aluminum hydroxyhalides (e.g., aluminum chlorohydrate) as well as mixtures and complexes thereof with zirconyl oxyhalides and zirconyl hydroxyhalides (e.g., aluminum zirconium chlorohydrate).

Preferred antiperspirant active ingredients can be chosen from water-soluble astringent organic and inorganic salts of aluminum, zirconium and zinc and/or any mixtures of these salts. Especially preferred antiperspirant active ingredients can be chosen from aluminum chlorohydrates, in particular aluminum chlorohydrates having the general formula [Al₂(OH)₅Cl.2-3H₂O]ₙ which may be present in activated (depolymerized) or unactivated form, as well as aluminum sesquichlorohydrate, aluminum chlorohydrex propylene glycol (PG) or polyethylene glycol (PEG), aluminum or aluminum zirconium-glycol complexes (e.g., aluminum or aluminum zirconium-propylene glycol complexes), aluminum sesquichlorohydrex PG or PEG, aluminum-PEG dichlorohydrex or aluminum PEG dichlorohydrex, aluminum hydroxide, also selected from aluminum zirconium chlorohydrates, such as aluminum zirconium trichlorohydrate, aluminum zirconium tetrachlorohydrate, aluminum zirconium pentachloro-hydrate, aluminum zirconium octachlorohydrate, aluminum zirconium chloro-hydrate-glycine complexes such as aluminum zirconium trichlorohydrex glycine, aluminum zirconium tetrachlorohydrex glycine, aluminum zirconium pentachlorohydrex glycine, aluminum zirconium octachlorohydrex glycine, potassium aluminum sulfate (KAl(SO₄)₂.12H₂O, alum), aluminum undecylenoyl collagen amino acid, sodium aluminum lactate+aluminum sulfate, sodium aluminum chlorohydroxylactate, aluminum bromohydrate, aluminum chloride, complexes of zinc and sodium salts, complexes of lanthanum and cerium, aluminum salts of lipoamino acids, aluminum sulfate, aluminum lactate, aluminum chlorohydroxyallantoinate, sodium aluminum chlorohydroxy-lactate, zinc chloride, zinc sulfocarbolate, zinc sulfate and zirconium chloro-hydrate. According to the invention, water solubility is understood to be a solubility of at least 5 wt % at 20° C. (i.e., amounts of at least 5 g of the antiperspirant active ingredient are soluble in 95 g water at 20° C.).

In an especially preferred embodiment, the composition contains an astringent aluminum salt, in particular aluminum chlorohydrate, which is distributed, for example, in the form of a powder under the names Micro Dry® Ultrafine, Micro Dry® Superultrafine, Chlorhydrol® Microdry and Chlorhydrol® Microdry Ultrafine, and in activated form as Reach® 501, Reach® 101 and Reach® 103 by Reheis. Also especially preferred are powdered aluminum sesquichlorohydrates such as Reach® 301 from Reheis or ACH-308 Alcohol Soluble Beads from Summit Research Labs. The use of aluminum zirconium tetrachlorohydrex glycine complexes such as those available commercially under the names Rezal® 36G Superultrafine or Reach® AZP 908 Powder Superultrafine may also be especially preferred according to the invention.

Other preferred aluminum salts include those with the general formula Al₂(OH)₆₋ₐXₐ, wherein X is Cl, Br, I or NO₃ and “a” is 0.3 to 4, preferably 1 to 2, so that the molar ratio of Al to X is in the range of 1:1 to 2.1:1. As a rule, these salts contain water of crystallization typically on the order of 1 to 6 mol water per mol salt. Most preferred is the aluminum salt aluminum chlorohydrate (i.e., X=Cl), wherein “a”=1 so that the molar ratio of aluminum to chlorine is 1.9:1 to 2.1:1.

Other preferred aluminum zirconium salts are mixtures or complexes of the aluminum salts described above with zirconium salts of the formula ZrO(OH)_{2-pb}Y_{b}, wherein Y is Cl, Br, I, NO₃ or SO₄, b is 0.8 to 2, and p denotes the valence of Y. The zirconium salts usually contain some water of crystallization, typically on the order of 1 to 7 mol water per mol salt. The zirconium salt is preferably zirconyl hydroxychloride of the formula ZrO(OH)_{2-b}Cl_{b}, wherein b is 1 to 2 and preferably 1.2 to 1.9. The preferred aluminum zirconium salts have an Al:Zr ratio of 1.7 to 12.5, most preferably 2 to 10, as well as a metal/(X+Y) ratio of 0.73 to 2.1, preferably 0.9 to 1.5. A preferred salt is aluminum zirconium chlorohydrate (i.e., X and Y are Cl), which has an Al:Zr ratio of 2 to 10 and a metal/Cl ratio of 0.9-2.1, preferably 0.95-1.5, especially preferably 1-1.3. The term “aluminum zirconium chlorohydrate” includes the forms tri-, tetra-, penta- and octachlorohydrate. Aluminum zirconium salt complexes may also contain a neutral amino acid, preferably glycine, typically with a Gly:Zr ratio of approximately 1:1, i.e., 0.8-1.2, especially preferably 1.

Preferred antiperspirant sticks may contain at least one antiperspirant active ingredient in an amount of 3-27 wt %, preferably 5-25 wt %, especially preferably 8-22 wt % and most especially preferably 18-20 wt %, based on total weight of the active substance (USP, US Pharmacopoeia) in the total composition.

The amount of antiperspirant salt(s), given in wt % in the present patent application, is calculated according to the method of the US Pharmacopoeia (USP), according to which the weight of the bound water of crystallization and other ligands (e.g., glycine) is not taken into account.

It has proven especially advantageous for rapid onset of effect and high efficacy if the astringent salts are coated or impregnated with a water-soluble nonionic surfactant. Especially suitable surfactants for this purpose include alkyl oligoglycosides. Coating or impregnation with alkyl oligoglycosides, which are applied to the surface of the salt particles in amounts of 0.1 to 5 wt %, is especially preferred.

In another especially preferred embodiment, the inventive stick compositions contain at least one deodorant and at least one antiperspirant active ingredient.

Water of crystallization added with the antiperspirant active ingredients is not taken into account in calculating the water content of the stick.

Preferred sticks according to the present invention contain a maximum amount of no more than 5 wt % water, especially preferably less than 3 wt % water, based on total weight of the stick composition. Water here refers to only free water, and not water bound as water of crystallization present in the antiperspirant active ingredients.

Preferred inventive antiperspirant sticks include those wherein the at least one linear polydimethylsiloxane having 2 to 50 siloxane units is chosen from hexamethyldisiloxane (L₂), octamethyltrisiloxane (L₃), decamethyltetra-siloxane (L₄), dodecamethylpentasiloxane (L₅), any mixtures of two, three or four of L₂, L₃, L₄ and/or L₅, linear polydimethylsiloxanes having 6 to 50 siloxane units, preferably 7-45 siloxane units, especially preferably 8-40 siloxane units, most especially preferably 9-35 siloxane units. Other preferred linear polydimethylsiloxanes are selected from those with 10, 11, 12, 13, 14, 15, 16, 17, 18, 19 and 20 siloxane units. Other preferred linear polydimethylsiloxanes are selected from those having 21, 22, 23, 24, 25, 26, 27, 28, 29 and 30 siloxane units. Other preferred linear polydimethylsiloxanes are selected from those having 31, 32, 33, 34, 35, 36, 37, 38, 39 and 40 siloxane units.

Corresponding linear polydimethylsiloxanes preferred according to the present invention are commercially available. The commercial products usually contain mixtures of linear polydimethylsiloxanes with different numbers of siloxane units. The specification of the commercial products tends to be based on the (kinematic) viscosity (at 25° C.) rather than the number of siloxane units. For example, hexamethyldisiloxane is commercially available as Dow Corning 200® Fluid, 0.65 CST, having a kinematic viscosity of 0.65 cSt (at 25° C.). Octamethyltrisiloxane, for example, is distributed as Dow Corning 200® Fluid, 1 CST, with a kinematic viscosity of 1 cSt (at 25° C.). Other commercial products with an average kinematic viscosity of 1.35-1.65 cSt (25° C.) are mixtures of linear polydimethylsiloxanes with different numbers of siloxane units. Dow Corning 200® Fluid, 1.5 CST, for example, contains primarily polydimethylsiloxanes with 3 and 4 siloxane units.

Corresponding linear polydimethylsiloxanes preferred according to the present invention are found, for example, in the commercial products DC 2-1184, Dow Corning® 200 (0.65 cSt), Dow Corning® 200 (1.0 cSt), Dow Corning 200® Fluid, 1.5 CST, Dow Corning 200® Fluid, 2 CST and Dow Corning 200® Fluid, 5 CST from Dow Corning.

Other linear polydimethylsiloxanes preferred according to the present invention include mixtures having a kinematic viscosity of 0.65-10 cSt (25° C.), preferably 1-9 cSt, especially preferably 1.5-8 cSt and most especially preferably 2-5 cSt.

The at least one linear polydimethylsiloxane having 2 to 50 siloxane units is preferably present in a total amount of 10-60 wt %, especially preferably 20-50 wt %, most especially preferably 25-35 wt %, based on total weight of the antiperspirant stick.

The inventive antiperspirant sticks especially preferably contain a mixture of linear polydimethylsiloxanes having a kinematic viscosity (25° C.) of 0.65-10 cSt, preferably 1-9 cSt, especially preferably 1.5-8 cSt and most especially preferably 2-5 cSt in a total amount of 10-60 wt %, especially preferably 20-50 wt %, most especially preferably 25-35 wt %, based on total weight of the total antiperspirant stick.

The inventive antiperspirant sticks also especially preferably contain two mixtures of linear polydimethylsiloxanes, each having a kinematic viscosity (25° C.) of 0.65-10 cSt, preferably 1-9 cSt especially preferably 1.5-8 cSt and most especially preferably 2-5 cSt in a total amount of 10-60 wt %, especially preferably 10-50 wt %, most especially preferably 25-35 wt %, based on total weight of the total antiperspirant stick. The combination of a mixture with 2 cSt and a mixture with 5 cSt is especially preferred here, especially in a weight ratio of 1:2 to 2:1, especially preferably 1:1.

The inventive antiperspirant sticks also especially preferably contain three mixtures of linear polydimethylsiloxanes each having a kinematic viscosity (25° C.) of 0.65-10 cSt, preferably 1-9 cSt, especially preferably 1.5-8 cSt and most especially preferably 2-5 cSt, in a total amount of 10-60 wt %, especially preferably 20-50 wt %, most especially preferably 25-35 wt %, based on the total antiperspirant stick.

The inventive antiperspirant sticks also especially preferably contain four mixtures of linear polydimethylsiloxanes, each having a kinematic viscosity (25° C.) of 0.65-10 cSt, preferably 1-9 cSt, especially preferably 1.5-8 cSt and most especially preferably 2-5 cSt in a total amount of 10-60 wt %, especially preferably 20-50 wt %, most especially preferably 25-35 wt %, based on the total antiperspirant stick.

Other preferred antiperspirant sticks according to the present invention do not contain any cyclomethicones.

Other antiperspirant sticks preferred according to the invention may contain at least one oil liquid under standard conditions chosen from poly-C₂-C₁₆-olefins, in particular chosen from polybutene, polyisobutene and polydecene.

In a preferred embodiment of the invention, the poly-C₂-C₁₆-olefin oils are present in hydrogenated form. Hydrogenated polybutene, hydrogenated polyisobutene and hydrogenated polydecene as well as mixtures thereof are especially preferred.

It is also preferred that the poly-C₂-C₁₆-olefin oils and the hydrogenated poly-C₂-C₁₆-olefin oils have a total C count of 20 to 60, preferably 24 to 50, especially preferably 30 to 40 in the molecule. Especially preferred are isoeicosane, hydrogenated polyisobutene (INCI designation) with at least 50 wt % C₃₂ content and hydrogenated polydecene (INCI designation) with 75-85 wt % C₃₀ content and 15-25 wt % C₄₀ content as well as mixtures thereof.

The at least one poly-C₂-C₁₆-olefin oil and/or the at least one hydrogenated poly-C₂-C₁₆-olefin oil is/are preferably present in a total amount of 5-30 wt %, especially preferably 10-25 wt %, most especially preferably 12-15 wt %, based on the total antiperspirant stick. Extremely preferred antiperspirant sticks are characterized in that they contain hydrogenated polydecene (INCI designation) with 75-85 wt % C₃₀ content and 15-25 wt % C₄₀ content in a total amount of 5-25 wt %, especially preferably 10-18 wt %, most especially preferably 12-15 wt %, based on total weight of the antiperspirant stick.

Other antiperspirant sticks preferred according to the invention may contain at least one linear polydimethylsiloxane with 2 to 50 siloxane units and at least one oil that is liquid under standard conditions, chosen from poly-C₂-C₁₆-olefins, in particular chosen from polybutene, polyisobutene and polydecene. The two substance classes (linear polydimethylsiloxane, polyolefin) are preferably present in a weight ratio of approximately 1:1, particularly when the linear polydimethylsiloxane is chosen from L₂ and/or L₃ and the polyolefin is chosen from polydecene.

The two substance classes (linear polydimethylsiloxane, polyolefin) are also preferably present in a weight ratio of approximately 1:1, particularly when the linear polydimethylsiloxane is chosen from those having 2 to 20 siloxane units, and the polyolefin is chosen from polydecene.

The two substance classes (linear polydimethylsiloxane, polyolefin) are also preferably present in a weight ratio of approximately 1:1, particularly when the linear polydimethylsiloxane is chosen from those having 2 to 20 siloxane units, and the polyolefin is chosen from hydrogenated polydecene (INCI designation) with 75-85 wt % C₃₀ content and 15-25 wt % C₄₀ content.

The two substance classes (linear polydimethylsiloxane, polyolefin) are also preferably present in a weight ratio of approximately 1:1, particularly when the linear polydimethylsiloxane is chosen from those having a kinematic viscosity (25° C.) of 0.65-10 cSt, preferably 1-9 cSt, especially preferably 1.5-8 cSt and most especially preferably 2-5 cSt, and the polyolefin is chosen from hydrogenated polydecene (INCI designation) with 75-85 wt % C₃₀ content and 15-25 wt % C₄₀ content.

Additional preferred antiperspirant sticks according to the invention are characterized in that the at least one lipid or wax component a) is chosen from coco fatty acid glycerol mono-, di- and triesters, Butyrospermum Parkii (shea butter), esters of saturated monovalent C₈-C₁₈ alcohols with saturated C₁₂-C₁₈ monocarboxylic acids, linear primary C₁₂-C₂₄ alkanols, esters of a saturated monovalent C₁₆-C₆₀ alkanol and a saturated C₈-C₃₆ monocarboxylic acid, in particular cetyl behenate, stearyl behenate and C₂₀-C₄₀ alkyl stearate, glycerol triesters of saturated linear C₁₂-C₃₀ carboxylic acids, which may be hydroxylated, candelilla wax, carnauba wax, beeswax, saturated linear C₁₄-C₃₆ carboxylic acids and mixtures of the aforementioned substances.

Said lipids and waxes cover a wide melting range. Those skilled in the art are aware that the mixture of lipid or wax components must be coordinated carefully with respect to their melting points and/or melting range for good abrasion and strength properties.

### Lipid or Wax Matrix

The lipid or wax matrix of the inventive stick compositions include at least one lipid or wax component having a melting point of >50° C.

In general, waxes have a solid to crumbly, hard consistency, are coarsely to finely crystalline, transparent to opaque but not vitreous and they melt above 50° C. without decomposing. They have a low viscosity, even above the melting point, and have a consistency and solubility which depend greatly on temperature.

Preferred waxes according to the invention include, for example, the natural plant-based waxes (e.g., candelilla wax, carnauba wax, Japan wax, sugar cane wax, ouricoury wax, cork wax, sunflower wax, fruit waxes such as orange wax, lemon wax, grapefruit wax) and animal waxes (e.g., beeswax, shellac wax and spermaceti). In the sense of the invention, it may be especially preferable to use hydrogenated or hardened waxes. Chemically modified waxes, in particular hard waxes (e.g. d montan ester waxes, hydrogenated jojoba waxes and sasol waxes) may also be used as the wax component. The synthetic waxes, which are also preferred according to the invention, include, for example, polyalkylene waxes and polyethylene waxes, C₂₀-C₄₀ dialkyl esters of dimeric acids, C₃₀₋₅₀ alkyl beeswax and alkyl and alkylaryl esters of dimeric fatty acids.

An especially preferred wax component is chosen from at least one ester of a saturated monovalent C₁₆-C₆₀ alcohol and a saturated C₈-C₃₆ monocarboxylic acid. According to the invention, these also include lactides, the cyclic double esters of α-hydroxycarboxylic acids with the corresponding chain length. Esters of fatty acids and long-chain alcohols have proven to be especially advantageous for the inventive composition because they impart excellent sensory properties to the antiperspirant preparation and impart a higher stability to the stick on the whole. The esters are composed of saturated, branched or unbranched monocarboxylic acids and saturated, branched or unbranched monovalent alcohols. Esters of aromatic carboxylic acids and/or hydroxycarboxylic acids (e.g., 12-hydroxystearic acid) and saturated, branched or unbranched alcohols can also be used according to the invention if the wax component has a melting point of >50° C. It is especially preferable to choose wax components from the group of esters of saturated, branched or unbranched alkanecarboxylic acids with a chain length of 12 to 24 carbon atoms and the saturated, branched or unbranched alcohols, which have a chain length of 16 to 50 carbon atoms and have a melting point of >50° C.

Especially advantageous wax components include C₁₆₋₃₆ alkyl stearates and C₁₈₋₃₈ alkylhydroxystearoyl stearates, C₂₀₋₄₀ alkyl erucates and cetearyl behenate. The wax or wax components have a melting point of >50° C., preferably >60° C., especially preferably in the range of 75-95° C., most especially preferably in the range of 80-90° C.

An especially preferred embodiment of the invention contains a C₂₀-C₄₀ alkyl stearate as the wax component. This ester is commercially known by the name Kester® wax K82H or Kester® wax K80H and is distributed by Koster Keunen Inc. This is the synthetic simulation of the monoester fraction of beeswax and is characterized by its hardness, oil gellability and broad compatibility with lipid components. This wax may be used as a stabilizer and consistency regulator for W/O and O/W emulsions. Kester wax offers the advantage that it has excellent oil gellability even at low concentrations and therefore makes the stick composition not too heavy and allows velvety rub-on application. Another especially preferred embodiment of the invention includes cetearyl behenate (i.e., mixtures of cetyl behenate and stearyl behenate) as the wax component. This ester is known by the name Kester® wax K62 and is distributed by Koster Keunen Inc.

Other preferred lipid or wax components with a melting point of >50° C. include triglycerides of saturated and optionally hydroxylated C₁₂₋₃₀ fatty acids such as hardened triglyceride fats (hydrogenated palm oil, hydrogenated coconut oil, hydrogenated castor oil), glyceryl tribehenate (tribehenol) or glyceryl tri-12-hydroxystearate, as well as synthetic whole esters of fatty acids and glycols or polyols with 2 to 6 carbon atoms as long as they have a melting point above 50° C. (e.g., preferably C₁₈-C₃₆ acid triglyceride (Syncrowax® HGL-C)).

According to the invention, the wax component is especially preferably hydrogenated castor oil commercially available as Cutina® HR with a melting point in the range of 85-88° C., for example.

Other preferred lipid or wax components with a melting point of >50° C. include the saturated linear C₁₄-C₃₆ carboxylic acids, in particular myristic acid, palmitic acid, stearic acid and behenic acid as well as mixtures of these compounds (e.g., Syncrowax® AW 1C (C₁₈-C₃₆ fatty acids) or Cutina® FS 45 (palmitic acid and stearic acid)).

Preferred inventive antiperspirant sticks include a wax component a) chosen from esters of a saturated monovalent C₁₂-C₆₀ alkanol and a saturated C₆-C₃₆ monocarboxylic acid, in particular lauryl laurate, lauryl myristate, lauryl palmitate, lauryl stearate, lauryl 12-hydroxystearate, lauryl eicosanate, lauryl behenate, lauryl lignocerate, lauryl cerate, lauryl myricate, myristyl laurate, myristyl myristate, myristyl palmitate, myristyl stearate, myristyl 12-hydroxystearate, myristyl eicosanate, myristyl behenate, myristyl lignocerate, myristyl cerate, myristyl myricate, cetyl laurate, cetyl myristate, cetyl palmitate, cetyl stearate, cetyl 12-hydroxystearate, cetyl eicosanate, cetyl behenate, cetyl lignocerate, cetyl cerate, cetyl myricate, stearyl laurate, stearyl myristate, stearyl palmitate, stearyl stearate, stearyl 12-hydroxystearate, stearyl eicosanate, stearyl behenate, stearyl lignocerate, stearyl cerate, stearyl myricate, 12-hydroxystearyl laurate, 12-hydroxystearyl myristate, 12-hydroxystearyl palmitate, 12-hydroxystearyl stearate, 12-hydroxy-stearyl 12-hydroxystearate, 12-hydroxystearyl eicosanate, 12-hydroxystearyl behenate, 12-hydroxystearyl lignocerate, 12-hydroxystearyl cerate, 12-hydroxy-stearyl myricate, arachyl laurate, arachyl myristate, arachyl palmitate, arachyl stearate, arachyl 12-hydroxystearate, arachyl eicosanate, arachyl behenate, arachyl lignocerate, arachyl cerate, arachyl myricate, behenyl laurate, behenyl myristate, behenyl palmitate, behenyl stearate, behenyl 12-hydroxystearate, behenyl eicosanate, behenyl behenate, behenyl lignocerate, behenyl cerate, behenyl myricate, lignoceryl laurate, lignoceryl myristate, lignoceryl palmitate, lignoceryl stearate, lignoceryl 12-hydroxystearate, lignoceryl eicosanate, ligno-ceryl behenate, lignoceryl lignocerate, lignoceryl cerate, lignoceryl myricate, ceryl laurate, ceryl myristate, ceryl palmitate, ceryl stearate, ceryl 12-hydroxystearate, ceryl eicosanate, ceryl behenate, ceryl lignocerate, ceryl cerate, ceryl myricate, myricyl laurate, myricyl myristate, myricyl palmitate, myricyl stearate, myricyl 12-hydroxystearate, myricyl eicosanate, myricyl behenate, myricyl lignocerate, myricyl cerate, myricyl myricate, especially preferably cetyl behenate, stearyl behenate and C₂₀-C₄₀ alkyl stearates, in particular arachyl stearate, behenyl stearate, lignoceryl stearate, ceryl stearate and myricyl stearate, also selected from glycerol triesters of saturated linear C₁₂-C₃₀ carboxylic acids, which may be hydroxylated, whereby these glycerol triester are preferably in the form of natural waxes, in particular candelilla wax, carnauba wax or beeswax or preferably in the form of natural oils, which may be fully hydrogenated (hardened), especially fully hydrogenated hardened castor oil (tri-12-hydroxystearol), tristearol, tribehenol, fully hydrogenated soybean oil, fully hydrogenated corn germ oil, fully hydrogenated sunflower oil, fully hydrogenated high-erucaic rapeseed oil (HEAR oil), fully hydrogenated low-erucaic rapeseed oil (LEAR oil), fully hydrogenated canola oil, fully hydrogenated crambe oil, fully hydrogenated oil of lady's smock, fully hydrogenated cottonseed oil, fully hydrogenated olive oil, fully hydrogenated thistle oil, fully hydrogenated sunflower oil, fully hydrogenated sesame oil, fully hydrogenated coconut oil, fully hydrogenated palm oil, fully hydrogenated palm kernel oil, fully hydrogenated babassu oil, fully hydrogenated peanut oil, fully hydrogenated cocoa butter, shea butter, illipe butter, hardened animal fats, in particular tallow or lard, fully hydrogenated oils of marine origin such as swordfish oil, sardine oil, sperm whale oil and herring oil,

Other preferred antiperspirant sticks include a wax component a) chosen from saturated linear C₁₄-C₃₆ carboxylic acids, in particular myristic acid, palmitic acid, stearic acid, 12-hydroxystearic acid, eicosanoic acid, behenic acid, lignoceric acid, ceric acid, myricic acid and mixtures of the aforementioned acids. Especially preferred wax component mixtures a) are selected from mixtures of cetyl behenate, stearyl behenate, hardened castor oil, palmitic acid and stearic acid. Other especially preferred wax component mixtures a) are chosen from mixtures comprising C₂₀-C₄₀ alkyl stearate, hardened castor oil, palmitic acid and stearic acid. Other especially preferred wax component mixtures a) are chosen from mixtures comprising C₁₆-C₂₀ alkyl behenate, hardened castor oil, palmitic acid and stearic acid. Other especially preferred wax component mixtures a) are chosen from mixtures comprising arachyl stearate, behenyl stearate, lignoceryl stearate, ceryl stearate, myricyl stearate, hardened castor oil, palmitic acid and stearic acid. Other especially preferred wax component mixtures a) are chosen from mixtures comprising palmityl behenate, stearyl behenate, arachyl behenate, hardened castor oil, palmitic acid and stearic acid.

### Low-Melting Lipid or Wax Component

Especially preferred inventive antiperspirant sticks include at least one lipid or wax component having a melting point in the range of >30° C. to <50° C., selected from coconut fatty acid glycerol mono-, di- and triesters, Butyrospermum Parkii (shea butter) and esters of saturated monovalent C₈-C₁₈ alcohols with saturated C₁₂-C₁₈ monocarboxylic acids and mixtures of these substances. These low-melting lipid or wax components allow the consistency of the product to be optimized and the visible residues on the skin to be minimized. Especially preferred commercial products are those with the INCI designation coco glycerides, in particular the commercial products Novata® (from Cognis), especially preferably Novata® AB, a mixture of C₁₂-C₁₈ mono-, di- and triglycerides that melts in the range of 30-32° C. as well as the products of the Softisan series (Sasol Germany GmbH) with the INCI designation hydrogenated coco glycerides, in particular Softisan 100, 133, 134, 138, 142. Other preferred esters of saturated monovalent C₁₂-C₁₈ alcohols with saturated C₁₂-C₁₈ monocarboxylic acids are stearyl laurate, cetearyl stearate (e.g. Crodamol® CSS), cetyl palmitate (e.g. Cutina® CP) and myristyl myristate (e.g. Cetiol® MM).

It has surprisingly been found that selected mixtures of certain lipid and wax components with a melting point of more than 30° C. lead to antiperspirant sticks having an especially balanced ratio of application behavior and/or spreadability, rub-on (=amount applied), stick hardness, dimensional stability and antiperspirant efficacy. Preferred lipid and wax components a) include in particular mixtures of i) at least one linear C₁₆-C₂₂ alkanol, selected from cetyl alcohol, stearyl alcohol, arachidyl alcohol and behenyl alcohol, ii) at least one glyceryl ester with a melting point in the range of 30-40° C., preferably in the range of 30-32° C., and iii) at least one wax or lipid with a melting point of more than 60° C., preferably in the range of 70-90° C. Especially preferred are mixtures of i), ii) and iii) in a weight ratio of i):ii):iii) of (15-20)-(3-6):(1-3) preferably (16-18):(4-5):(1.5-2). In addition, it is especially preferred if i) stearyl alcohol is used as the linear C₁₆-C₂₂ alkanol; cetyl alcohol is less preferred because larger amounts of cetyl alcohol do not adhere to the skin so well and may form a slightly crumbly residue. It is especially preferred if, in addition to stearyl alcohol, small amounts of behenyl alcohol, in particular 0.1-0.2 wt % are added.

Such mixtures i), ii) and iii) yield preferred antiperspirant sticks according to the invention having good rub-on properties, excellent residue behavior, a very good antiperspirant effect and good strength.

Preferred inventive antiperspirant sticks are characterized in that the lipid or wax component a) is present in a total amount of 18-30 wt %, preferably 20-25 wt %, especially preferably 22-24 wt %, each based on total weight of the antiperspirant stick.

It has surprisingly been found that the antiperspirant effect and/or the application properties of the inventive antiperspirant sticks can be further improved by adding an oil-in-water emulsifier. Nonionic oil-in-water emulsifiers are preferred for reasons of skin tolerability in particular.

Other preferred inventive antiperspirant sticks include those containing at least one oil-in-water emulsifier, preferably nonionic.

Oil-in-water emulsifiers having a melting point of more than 30° C. are not included with the claimed lipid or wax component a).

Substantially all surfactants that are soluble in the system in the amount of 1 wt % at 20° C. and are soluble in water at 20° C. in the amount of at least 1 wt % are basically suitable as the water-soluble surfactants. Although the structure and ionogenicity are essentially irrelevant, nonionic surfactants, in particular the addition products of ethylene oxide onto fatty substance molecules having at least one alkoxylatable group, where the addition products are solid under standard conditions, seem to be preferred. Such suitable surfactants include, for example, the addition products of 10-40 mol ethylene oxide onto linear fatty alcohols with 16-22 carbon atoms, onto fatty acids with 12-22 carbon atoms, onto fatty acid alkanolamides, onto fatty acid monoglycerides, onto sorbitan fatty acid monoesters, onto fatty acid alkanolamides, onto fatty acid glycerides, e.g. onto hardened castor oil, onto methyl glucoside monofatty acid esters and mixtures thereof.

The nonionic oil-in-water emulsifier is especially preferably chosen from surfactant substances with an HLB value of more than 7, such as:
ethoxylated C₈-C₂₄ alkanols with an average of 10-100 mol ethylene oxide per mol,
ethoxylated C₈-C₂₄ carboxylic acids with an average of 10-100 mol ethylene oxide per mol,
silicone copolyols with ethylene oxide units or with ethylene oxide and propylene oxide units,
alkyl mono- and oligoglycosides with 8 to 22 carbon atoms in the alkyl radical and their ethoxylated analogs,
ethoxylated sterols,
partial esters of polyglycerols with n=2 to 10 glycerol units and esterified with 1 to 4 saturated or unsaturated, linear or branched, optionally hydroxylated C₈-C₃₀ fatty acid radicals, if they have an HLB value of more than 7,
as well as mixtures of the aforementioned substances.

Preferred inventive antiperspirant sticks include nonionic oil-in-water emulsifiers chosen from ethoxylated C₈-C₂₄ alkanols with an average of 10-100 mol ethylene oxide per mol, ethoxylated C₈-C₂₄ carboxylic acids with an average of 10-100 mol ethylene oxide per mol, silicone copolyols with ethylene oxide units or with ethylene oxide and propylene oxide units, alkyl mono- and oligoglycosides with 8 to 22 carbon atoms in the alkyl radical and their ethoxylated analogs, ethoxylated sterols, partial esters of polyglycerols with n=2 to 10 glycerol units and esterified with 1 to 4 saturated or unsaturated, linear or branched optionally hydroxylated C₈-C₃₀ fatty acid radicals, if they have an HLB value of more than 7, as well as mixtures of the aforementioned substances

Ethoxylated C₈-C₂₄ alkanols have the formula R¹O(CH₂CH₂O)ₙH where R¹ stands for a linear or branched alkyl and/or alkenyl radical with 8 to 24 carbon atoms, and n, which denotes the average number of ethylene oxide units per molecule, stands for numbers from 10 to 100, preferably 10 to 30 mol ethylene oxide onto 1 mol capryl alcohol, 2-ethylhexyl alcohol, caprin alcohol, lauryl alcohol, isotridecyl alcohol, myristyl alcohol, cetyl alcohol, palmitoleyl alcohol, isocetyl alcohol, stearyl alcohol, isotridecyl alcohol, oleyl alcohol, elaidyl alcohol, petroselinyl alcohol, arachyl alcohol, gadoleyl alcohol, behenyl alcohol, erucyl alcohol, brassidyl alcohol and cetearyl alcohol as well as their technical-grade mixtures. Adducts of 10 to 100 mol ethylene oxide onto technical-grade fatty alcohols with 12 to 18 carbon atoms such as coco, palm, palm kernel or tallow fatty alcohol are suitable.

Ethoxylated C₈-C₂₄ carboxylic acids have the formula R¹O(CH₂CH₂O)ₙH, where R¹O stands for a linear or branched, saturated or unsaturated acyl radical with 8-24 hydrocarbon atoms, and n, which denotes the average number (weight average) of ethylene oxide units per molecule, stands for numbers from 10 to 100, preferably 10 to 50, especially preferably 30 to 40 mol ethylene oxide onto 1 mol caprylic acid, 2-ethylhexanoic acid, capric acid, lauric acid, isotridecanoic acid, myristic acid, cetylic acid, palmitoleic acid, stearic acid, isostearic acid, oleic acid, elaidic acid, petroselinic acid, arachyinic acid, gadolenic acid, behenic acid, erucaic acid and brassidic acid and their technical-grade mixtures. Adducts of 10-100 mol ethylene oxide onto technical-grade fatty acids with 12-18 carbon atoms such as coco, palm, palm kernel or tallow fatty acids are suitable. Especially preferred are PEG-40 monostearate, PEG-50 monostearate, PEG-100 monostearate, PEG-40 monooleate, PEG-50 monooleate, PEG-100 monooleate, PEG-40 monolaurate, PEG-50 mono-laurate and PEG-100 monolaurate.

C₁₂-C₁₈ alkanols or C₁₂-C₁₈ carboxylic acids, each with 10 to 40 units of ethylene oxide per molecule, as well as mixtures of these substances are especially preferably used, in particular ceteth-12, ceteth-20, isoceteth-20, ceteth-30, steareth-12, steareth-20, steareth-30, ceteareth-12, ceteareth-20, ceteareth-30, laureth-12, beheneth-20 and PEG-40 monostearate.

In addition, C₈-C₂₂ alkyl mono- and oligoglycosides are preferably used. C₈-C₂₂ alkyl mono- and oligoglycosides are known commercial surfactants and emulsifiers. They are synthesized by reaction of glucose or oligosaccharides with primary alcohols having 8-22 carbon atoms in particular. With regard to the glycoside radical, both monoglycosides in which a cyclic sugar radical is glycosidically bound to the fatty alcohol and oligomeric glycosides with a degree of oligomerization of up to approximately 8, preferably 1-2, are suitable. The degree of oligomerization is a statistical average based on a conventional homolog distribution for such technical-grade products. Products obtainable under the brand name Plantacare® contain a glucosidically bound C₈-C₁₆ alkyl group on an oligoglucoside radical whose average degree of oligomerization is 1-2. Especially preferred C₈-C₂₂ alkyl mono- and oligoglycosides are selected from octyl glucoside, decyl glucoside, lauryl glucoside, palmityl glucoside, isostearyl glucoside, stearyl glucoside, arachidyl glucoside and behenyl glucoside as well as mixtures thereof. The acyl glucamides derived from glucamine are also suitable as nonionic oil-in-water emulsifiers.

Ethoxylated sterols, in particular ethoxylated soy sterols, are suitable oil-in-water emulsifiers according to the invention. The degree of ethoxylation may be greater than 5, preferably at least 10, to have an HLB value greater than 7. Suitable commercial products include, for example, PEG-10 soy sterol, PEG-16 soy sterol and PEG-25 soy sterol.

In addition, partial esters of polyglycerols with 2 to 10 glycerol units and esterified with 1 to 4 saturated or unsaturated linear or branched, optionally hydroxylated C₈-C₃₀ fatty acid radicals are preferably also used, if they have an HLB value of more than 7. Especially preferred are diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate and decaglycerol trihydroxystearate.

Other especially preferred inventive antiperspirant sticks include at least one oil-in-water emulsifier present in a total amount of 0.5-10 wt %, preferably 1-4 wt %, most especially preferably 2-3 wt %, based on total weight of the stick.

It has surprisingly been found that the antiperspirant effect of the inventive antiperspirant sticks can be further improved by adding a polar oil.

Other preferred inventive antiperspirant sticks include a polar oil that is liquid under standard conditions and chosen from:
addition products of at least 6 ethylene oxide units and/or propylene oxide units onto monovalent or polyvalent C₃₋₂₂ alkanols,
addition products of 1 to 5 propylene oxide units onto monovalent or polyvalent C₈₋₂₂ alkanols,
C₈-C₂₂ fatty alcohol ethers,
C₈-C₂₂ fatty alcohol esters of monovalent or polyvalent C₂-C₇ hydroxycarboxylic acids,
branched, saturated or unsaturated fatty alcohols with 6-30 carbon atoms,
symmetrical, asymmetrical or cyclic esters of carbonic acid with C₈-C₂₂ fatty alcohols, in particular di-n-octyl carbonate, di-2-ethylhexyl carbonate and didodecyl carbonate,
triglycerides of linear or branched, saturated or unsaturated optionally hydroxylated C₈₋₃₀ fatty acids,
dicarboxylic acid esters of linear or branched C₂-C₁₀ alkanols,
esters of branched, saturated or unsaturated fatty alcohols with 2-30 carbon atoms with linear or branched, saturated or unsaturated fatty acids with 2-30 carbon atoms, which may be hydroxylated,
esters of dimers of unsaturated C₁₂-C₂₂ fatty acids (dimer fatty acids) with monovalent linear or branched or cyclic C₂-C₁₈ alkanols or with polyvalent linear or branched C₂-C₆ alkanols,
as well as mixtures of the aforementioned substances.

Especially preferred polar oils are chosen from addition products of at least 6 ethylene oxide units and/or propylene oxide units onto monovalent or polyvalent C₃₋₂₂ alkanols, such as butanol, butanediol, myristyl alcohol and stearyl alcohol, e.g. PPG-14 butyl ether (Ucon Fluid® AP), PPG-9 butyl ether (Breox® B25), PPG-10 butanediol (Macol® 57) and PPG-15 stearyl ether (Arlamol® E).

Additional polar oils that are especially preferred according to the invention are chosen from addition products of 1 to 5 propylene oxide units onto monovalent or polyvalent C₈₋₂₂ alkanols, such as octanol, decanol, decanediol, lauryl alcohol, myristyl alcohol and stearyl alcohol, e.g. PPG-2 myristyl ether and PPG-3 myristyl ether (Witconol® APM).

Other polar oils that are also very preferred are chosen from C₈₋₂₂ fatty alcohol ethers, in particular di-n-octyl ether, di-2-ethylhexyl ether and didodecyl ether.

Other polar oils that are also very preferred are chosen from C₈₋₂₂ fatty alcohol esters of monovalent or polyvalent C₂-C₇ hydroxycarboxylic acids, in particular the esters of glycolic acid, lactic acid, malic acid, tartaric acid, citric acid and salicylic acid. Such esters based on linear C_{14/15} alkanols, e.g. C₁₂-C₁₅ alkyl lactate and Cl_{12/13} alkanols with branching in position 2 are available under the brand name Cosmacol® from the company Nordmann, Rassmann GmbH and Co., Hamburg, in particular the commercial products Cosmacol® ESI, Cosmacol® EMI and Cosmacol® ETI.

Other polar oils preferred according to the invention are chosen from branched, saturated or unsaturated fatty alcohols with 6-30 carbon atoms. These alcohols are often also referred to as Guerbet alcohols because they are obtainable by the Guerbet reaction. Preferred alcohol oils are hexyldecanol (Eutanol® G 16, Guerbitol® T 16), octyldodecanol (Eutanol® G, Guerbitol® 20), 2-ethylhexyl alcohol and the commercial products Guerbitol® 18, Isofol® 12, Isofol® 16, Isofol® 24, Isofol® 36, Isocarb® 12, Isocarb® 16 or Isocarb® 24.

Other preferred polar oils according to the invention include mixtures of Guerbet alcohols and Guerbet alcohol esters (e.g., the commercial products Cetiol® PGL (hexyldecanol and hexyldecyl laurate)). Other preferred polar oils according to the invention are chosen from symmetrical, asymmetrical or cyclic esters of carbonic acid with fatty alcohols (e.g., glycerol carbonate, dicaprylyl carbonate (Cetiol® CC)) or esters according to DE 197 56 454 A1.

Other suitable polar oils according to the invention are chosen from triglycerides of linear or branched, saturated or unsaturated optionally hydroxylated C₈₋₃₀ fatty acids. Especially suitable may be the use of natural oils (e.g., soy oil, cottonseed oil, sunflower oil, palm oil, palm kernel oil, linseed oil, almond oil, castor oil, corn oil, olive oil, rapeseed oil, sesame oil, thistle oil, wheat germ oil, peach pit oil and the liquid fractions of coconut oil and the like). However, synthetic triglycerides, in particular capric/caprylic triglycerides are also suitable (e.g., the commercial products Myritol® 318, Myritol® 331 (Cognis) or Miglyol® 812 (Hüls) with unbranched fatty acid radicals as well as glyceryl triisostearol and the commercial products Estol® GTEH 3609 (Uniqerna) or Myritol® GTEH (Cognis) with branched fatty acid radicals).

Other suitable polar oils according to the invention are chosen from dicarboxylic acid esters of linear or branched C₂-C₁₀ alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicapryl maleate, diisooctyl succinate, di-2-ethylhexyl succinate and di-(2-hexyldecyl) succinate.

Other suitable polar oils according to the invention are chosen from esters of linear or branched, saturated or unsaturated fatty alcohols with 2-30 carbon atoms with linear or branched, saturated or unsaturated fatty acids with 2-30 carbon atoms, which may be hydroxylated. These include hexyldecyl stearate (Eutanol® G 16 S), hexyldecyl laurate, isodecyl neopentanoate, isononylisononanoate, 2-ethylhexyl palmitate (Cegesoft® C 24) and 2-ethylhexyl stearate (Cetiol® 868). Also suitable to a limited extend are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and dipalmitate.

Other suitable polar oils according to the invention are chosen from esters of dimers of unsaturated Cl₁₂₋₂₂ fatty acids (dimer fatty acids) with monovalent linear, branched or cyclic C₂-C₁₈ alkanols or with polyvalent linear or branched C₂-C₆ alkanols.

It may be preferable according to the invention to use mixtures of the aforementioned oils.

Especially preferred inventive antiperspirant sticks include at least one polar oil, in particular at least one of the polar oils listed above, present in a total amount of 2-25 wt %, preferably 5-20 wt %, especially preferably 7.5-15 wt % and most especially preferably 10-12 t %, based on total weight of the antiperspirant stick.

In addition to the linear polydimethylsiloxanes with 2 to 50 siloxane units, optionally the poly-C₂-C₁₆-olefins oils, preferably hydrogenated, and optionally the polar oils, antiperspirants sticks that are preferred according to the present invention may also contain at least one phenyl-substituted polydimethyl-siloxane, in particular phenyl trimethicone. Phenyl-substituted polydimethyl-siloxanes are preferably present in amounts of 0.1-3 wt % and promote the residue behavior and application properties. They may also further optimize the antiperspirant efficacy.

Other preferred antiperspirant sticks may contain at least one deodorant active ingredient, preferably selected from:
arylsulfatase inhibitors, β-glucuronidase inhibitors, aminoacylase inhibitors, esterase inhibitors, lipase inhibitors and lipoxigenase inhibitors,
α-monoalkyl glycerol ethers with a branched or linear, saturated or unsaturated, optionally hydroxylated C₆-C₂₂ alkyl radical, in particular α-(2-ethylhexyl) glycerol ether,
phenoxyethanol,
antibacterial perfume oils,
Deosafe perfume oils,
prebiotic active components,
trialkyl citric acid esters, in particular triethyl citrate,
active ingredients that reduce the number of and/or inhibit the growth of skin microorganisms that cause body odor, from the group of staphylococci, corynebacteria, anaerococci and micrococci,
zinc compounds, in particular zinc phenolsulfonate and zinc ricin-oleate,
organohalogen compounds, in particular triclosan, chlorhexidine, chlorhexidine gluconate and benzalkonium halides,
quaternary ammonium compounds, in particular cetylpyridinium chloride,
odor absorbers, in particular silicates and zeolites,
sodium bicarbonate,
lantibiotics,
as well as mixtures of the aforementioned substances,
   and/or the antiperspirant active ingredient is chosen from water-soluble astringent, organic and inorganic salts of aluminum, zirconium and zinc and/or any mixtures of these salts.

Other preferred inventive antiperspirant sticks are characterized in that the deodorant active ingredient is present in a total amount of 0.1-10 wt %, preferably 0.2-7 wt %, especially preferably 0.3-5 wt % and most especially preferably 0.4-10 wt %, based on 0.1-10 wt %, each based on total weight of the active substance in the inventive total composition.

In addition, the inventive stick compositions may contain the usual additives and cosmetic and/or dermatological active ingredients.

Inert finely divided organic or inorganic fillers are especially important here. Such inert fillers may include, for example, silicic acids, clays, talc, veegum or organic fillers, e.g. polymer powder, starch or cellulose powder. The inventive antiperspirant stick compositions preferably also contain 1-20 wt % of finely divided filler, selected from talc, silicic acid and mixtures thereof. Through such fillers, the strength, oil binding capacity and rub-on behavior of the stick are definitely improved.

Other preferred inventive antiperspirant sticks may also contain at least one solid water-insoluble particulate filler.

Especially preferred solid water-insoluble particulate fillers are chosen from optionally modified starches and starch derivatives, which are pregelatinated, if desired, cellulose and cellulose derivatives, silicon dioxide, silicic acids, spherical polyalkylsesquioxane particles, silica gels, talc, kaolin, clays, e.g. bentonites, magnesium aluminum silicates, boron nitride, lactoglobulin derivatives, glass powder, polymer powders and mixtures of the aforementioned substances.

Other preferred inventive antiperspirant sticks include the at least one solid water-insoluble particulate filler in a total amount of 0.01 to 20 wt %, preferably 5 to 15 wt %, based on weight of the total composition.

Other additives that are customary in deodorant and antiperspirant compositions and that may be added include perfumes, antioxidants, chelating agents, antimicrobial substances and odor-absorbing polymers. Finally, dyes and colored or white pigments may also be added to increase the attractiveness of the sticks and at least one hair growth-inhibiting substance may also be added.

The present invention also provides a cosmetic nontherapeutic method for reducing body odor wherein an antiperspirant stick composition such as that described above, in particular an antiperspirant stick composition according to exemplary embodiments 1-16, and in particular an antiperspirant stick composition according to any one of claims, is applied to the skin, in particular the underarm skin.

Another subject of the present invention is the nontherapeutic use of an antiperspirant stick composition such as that described above, in particular an antiperspirant stick composition according to exemplary embodiments 1-16 and in particular an antiperspirant stick composition according to any one of claims for reducing body odor.

The following examples should illustrate the subject of the present invention in greater detail without restricting it thereto.

Examples 1-6 are extremely preferred antiperspirant sticks according to the invention with good abrasion, excellent residue behavior, very good antiperspirant effect and good strength.

All quantitative amounts are based on weight percent. The quantitative amounts for the antiperspirant active ingredient are based on wt % raw material as is.

| | 1 | 2 | 3 | 4 | 5 | 6 |
|---|---|---|---|---|---|---|
| Lorol C18 | 16 | 18 | 18 | 18 | 18 | 18 |
| Lanette 22 | — | — | 0.2 | — | — | — |
| Eumulgin B3 | 3 | 3 | 3 | 3 | 3 | 3 |
| Ucon Fluid AP | 15 | 5 | 10 | 15 | 10 | 7.5 |
| Silicone oil DC 200 2 cSt | — | — | 18.4 | 23 | 24.5 | 25 |
| Silicone oil DC 200 5 cSt | 33.5 | 44.5 | 18.4 | 11.5 | — | — |
| Nexbase 2004 | — | — | — | — | 15 | 15 |
| Novata AB | 5 | 4 | 4 | 4 | 4 | 4 |
| Cutina HR | 1.5 | 1.5 | 2 | 1.5 | 1.5 | 1.5 |
| ACH Micro Dry UF | 20 | 20 | 20 | 20 | 20 | 20 |
| Talc Pharma G | 5 | 3 | 5 | 3 | 3 | 5 |
| Perfume | 1 | 1 | 1 | 1 | 1 | 1 |

Examples 7-16 represent inventive antiperspirant sticks having good rub-on behavior, good residue behavior, very good antiperspirant effect and satisfactory strength.

All quantitative amounts are based on wt %. The quantitative amounts for the antiperspirant active ingredient are based on wt % (USP).

| | 7 | 8 | 9 | 10 | 11 | 12 | 13 | 14 | 15 | 16 |
|---|---|---|---|---|---|---|---|---|---|---|
| Silicone oil DC 2-1184 | 28 | 28 | 23 | 23 | 23 | — | — | — | — | — |
| (Dow Corning) | | | | | | | | | | |
| Silicone oil Dow | — | — | — | — | — | 14 | 14 | 11 | 11 | 23 |
| Corning ® | | | | | | | | | | |
| 200 (0.65 cSt) | | | | | | | | | | |
| Polydecene | — | — | — | — | — | 14 | 14 | 11 | 11 | — |
| (Nexbase 2004) | | | | | | | | | | |
| Eutanol ® G 16 | 10 | — | — | 15 | 10 | 10 | — | — | 15 | 10 |
| Cetiol ® OE | — | 10 | 15 | — | — | — | 10 | 15 | — | — |
| Ucon Fluid ® AP | 5 | 5 | 5 | 5 | 5 | 5 | 5 | 5 | 5 | 5 |
| Cutina ® HR | 6 | 6 | 6 | 6 | 6 | 6 | 6 | 6 | 6 | 6 |
| Lorol ® C 18 | 20 | 20 | 20 | 20 | 20 | 20 | 20 | 20 | 20 | 20 |
| Eumulgin ® B 3 | 3 | 3 | 3 | 3 | 3 | 3 | 3 | 3 | 3 | 3 |
| Cutina ® E 24 PF | — | — | — | — | 5 | — | — | — | — | 5 |
| ACH | 20 | 20 | 20 | 20 | 20 | 20 | 20 | 20 | 20 | 20 |
| Talc | 8 | 8 | 8 | 8 | 8 | 8 | 8 | 8 | 8 | 8 |

The following commercial products were used to produce the inventive antiperspirant sticks:

| | |
|---|---|
| ACH Micro Dry ® UF | Aluminum chlorohydrate (Reheis/Interorgana) |
| (Ultrafine) | |
| Cetiol ® OE | Di-n-octyl ether (Cognis) |
| Cutina ® E 24 PF | PEG-20 glyceryl stearate(Cognis) |
| Cutina ® HR | Hydrogenated castor oil (Cogis) |
| Eumulgin ® B3 | Ceteareth-30 (EG-30-cetyl/stearyl ether) |
| | (Cognis) |
| Eutanol ® G16 | 2-Hexyldecanol (Cognis) |
| Lanette ® 22 | Behenyl alcohol (Cognis) |
| Lorol ® C 18 | Stearyl alcohol (Cognis) |
| Nexbase 2004 | Hydrogenated polydecene (Nestle) with 75-85 |
| | wt % C₃₀ fraction and 15-25 wt % C₄₀ fraction |
| Novata ® AB | Coco glycerides (Cognis) |
| Silicone oil DC 200 | Dimethicone (0.65 cSt) = hexamethyldisiloxane |
| 0.65 cSt | (Dow Corning) |
| Silicone oil DC 200 2 cSt | Dimethicone (2 cSt) (Dow Corning) |
| Silicone oil DC 200 5 cSt | Dimethicone (5 cSt) (Dow Corning) |
| Silicone oil DC 2-1184 | Trisiloxane, dimethicone (Dow Corning) |
| Talc Pharma G | Talc |
| Ucon Fluid ® AP | PPG-14 butyl ether |

We claim:

1. Anhydrous antiperspirant stick comprising:
at least one lipid or wax component having a melting point of >30° C.,
at least one oil that is liquid under standard conditions and is chosen from at least one linear polydimethylsiloxane with 2 to 50 siloxane units,
up to 5 wt % water, based on total weight of the antiperspirant stick, and
at least one antiperspirant active ingredient.

2. Antiperspirant stick according to claim 1, wherein it does not contain any cyclomethicone.

3. Antiperspirant stick according to claim 1, wherein the at least one linear polydimethylsiloxane with 2 to 50 siloxane units is present in an amount of 10-60 wt %, based on total weight of the antiperspirant stick.

4. Antiperspirant stick according to claim 1, wherein the at least one linear polydimethylsiloxane with 2 to 50 siloxane units is chosen from hexamethyldisiloxane (L₂), octamethyltrisiloxane (L₃), decamethyltetrasiloxane (L₄), dodecamethylpentasiloxane (L₅) or any mixtures of two, three or four L₂, L₃, L₄ and/or L₅.

5. Antiperspirant stick according to claim 1, wherein the at least one oil that is liquid under standard conditions is chosen from at least one poly-C₂-C₁₆-olefin oil which may be present in hydrogenated form.

6. Antiperspirant stick according to claim 5 further comprising the at least one poly-C₂-C₁₆-olefin oil and/or the at least one hydrogenated poly-C₂-C₁₆-olefin oil in an amount of 5-30 wt %, based on total weight of the antiperspirant stick.

7. Antiperspirant stick according to claim 1, wherein the lipid or wax component is chosen from coco fatty acid glycerol mono-, di- and triesters, Butyrospermum Parkii (shea butter), esters of saturated monovalent C₈-C₁₈ alcohols with saturated C₁₂-C₁₈ monocarboxylic acids, linear primary C₁₂-C₂₄ alkanols, esters of a saturated monovalent C₁₆-C₆₀ alkanol and a saturated C₈-C₃₆ monocarboxylic acid, in particular cetyl behenate, stearyl behenate and C₂₀-C₄₀ alkyl stearate, glycerol triesters of saturated linear C₁₂-C₃₀ carboxylic acids, which may be hydroxylated, candelilla wax, carnauba wax, beeswax, saturated linear C₁₄-C₃₆ carboxylic acids as well as mixtures of the aforementioned substances.

8. Antiperspirant stick according to claim 1, wherein the lipid and wax component is chosen from mixtures of at least one linear C₁₆-C₂₂ alkanol, at least one glyceryl ester with a melting point in the range of 30-40° C., and at least one wax or lipid with a melting point of greater than 60° C.

9. Antiperspirant stick according to claim 8, wherein it contains a mixture of alkanol, ester and wax or lipid in a weight ratio of (15-20):(3-6):(1-3).

10. Antiperspirant stick according to claim 1, wherein the lipid or wax component is present in an amount of 18-30 wt %, based on total weight of the antiperspirant stick.

11. Antiperspirant stick according to claim 1 further comprising at least one oil-in-water emulsifier.

12. Antiperspirant stick according to claim 11, wherein the oil-in-water emulsifier is nonionic and chosen from surfactant substances having an HLB value of more than 7.

13. Antiperspirant stick according to claim 12, wherein the surfactant substances having an HLB value of more than 7 are selected from ethoxylated C₈-C₂₄ alkanols with an average of 10-100 mol ethylene oxide per mol, ethoxylated C₈-C₂₄ carboxylic acids with an average of 10-100 mol ethylene oxide per mol, silicone copolyols with ethylene oxide units or with ethylene oxide and propylene oxide units, alkyl mono- and oligoglycosides with 8 to 22 carbon atoms in the alkyl radical and their ethoxylated analogs, ethoxylated sterols, partial esters of polyglycerols with n=2 to 10 glycerol units and esterified with 1 to 4 saturated or unsaturated, linear or branched, optionally hydroxylated C₈-C₃₀ fatty acid radicals having an HLB value of more than 7, as well as mixtures of the aforementioned substances.

14. Antiperspirant stick according to claim 11, wherein the oil-in-water emulsifier is present in an amount of 0.5-10 wt %, based on total weight of the antiperspirant stick.

15. Antiperspirant stick according to claim 1 further comprising a polar oil that is liquid under standard conditions and chosen from addition products of at least 6 ethylene oxide units and/or propylene oxide units onto monovalent or polyvalent C₃₋₂₂ alkanols, addition products of 1 to 5 propylene oxide units onto monovalent or polyvalent C₈₋₂₂ alkanols, C₈-C₂₂ fatty alcohol ethers, C₈-C₂₂ fatty alcohol esters of monovalent or polyvalent C₂-C₇ hydroxycarboxylic acids, branched, saturated or unsaturated fatty alcohols with 6-30 carbon atoms, symmetrical, asymmetrical or cyclic esters of carbonic acid with C₈-C₂₂ fatty alcohols, in particular di-n-octyl carbonate, di-2-ethylhexyl carbonate and didodecyl carbonate, triglycerides of linear or branched, saturated or unsaturated, optionally hydroxylated C₈₋₃₀ fatty acids, dicarboxylic acid esters of linear or branched C₂-C₁₀ alkanols, esters of branched, saturated or unsaturated fatty alcohols with 2-30 carbon atoms with linear or branched, saturated or unsaturated fatty acids with 2-30 carbon atoms, which may be hydroxylated, the esters of dimers of unsaturated C₁₂-C₂₂ fatty acids (dimer fatty acids) with monovalent linear or branched or cyclic C₂-C₁₈ alkanols or with polyvalent linear or branched C₂-C₆ alkanols, as well as mixtures of the aforementioned substances.

16. Antiperspirant stick according claim 1, wherein the antiperspirant active ingredient is present in an amount of 3-27 wt %, based on total weight of the active substance (USP).

17. Antiperspirant stick according claim 1 further comprising at least one solid water-insoluble particulate filler.

18. Antiperspirant stick according claim 17, wherein the solid water-insoluble particulate filler is chosen from optionally modified starches and starch derivatives, cellulose and cellulose derivatives, silicon dioxide, silicic acids, spherical polyalkyl sesquioxane particles, silica gels, talc, kaolin, clays, magnesium aluminum silicates, boron nitride, lactoglobulin derivatives, glass powder, polymer powders and mixtures of the aforementioned substances.

19. Antiperspirant stick according to claim 17, wherein the solid water-insoluble particulate filler is present in an amount of 0.01 to 20 wt %, based on total weight of the antiperspirant stick.

20. Cosmetic nontherapeutic method for reducing body odor comprising applying an antiperspirant stick composition according claim 1 to the skin.
